(19) United States
(12) Patent Application Publication
(10) Pub. No.: US 2008/0145440 A1
(43) Pub. Date: Jun. 19, 2008

(54) Microparticle preparation and medical composition

(75) Inventors: Han Hee Cho, Kyoto-shi (JP); Long Hai Han, Kyoto-shi (JP); Kazuaki Matsumura, Kyoto-shi (JP); Sadami Tsutsumi, Kyoto-shi (JP); Suong Hyu Hyon, Uji-shi (JP); Masaji Matsunaga, Eniwa-shi (JP); Fumihito Yoshida, Eniwa-shi (JP)

Correspondence Address: Armstrong, Kratz, Quintos,;Hanson & Brooks, LLP, Suite 1000, 1725 K Street, NW, Washington, DC, 20006, US

(73) Assignees: NISSEI BIO COMPANY, LIMITED, Eniwa-shi (JP); Suong Hyu Hyon, Uji-shi (JP)

(21) Appl. No.: 11/812,468

(22) Filed: Jun. 19, 2007

(30) Foreign Application Priority Data
Dec. 19, 2006 (JP) 2006-341244

(51) Int. Cl. A61K 33/24; A61K 31/282; A61K 47/30; A61K 9/14; A61P 35/00

(52) U.S. Cl. 424/499; 424/649; 514/492; 514/777

(57) ABSTRACT

According to the present invention, an early-phase release amount of cisplatin (CCDP) is reduced and a release of cisplatin is maintained over a long period of time, such that an anticancer effect against cancer cells is enhanced while side effects are reduced. In this manner, a life prolongation effect for patients is enhanced. A microparticle preparation contains cisplatin that is intercalated into double-helical DNA. The microparticle preparation stably retains cisplatin and exhibits an excellent slow-release property. Accordingly, even when cisplatin containing severe toxicity is used, the side effects can be suppressed and an excellent anticancer effect can be provided over a long period of time.

The entire disclosure of Japanese Patent Application No. 2006-341244, filed Dec. 19, 2006, and Han Hee CHO et. al, “Drug Delivery System Using Complex of Anticancer Agent (Cis-platin) and Salmon-Derived DNA”, Official Journal of the Japan Society of Drug Delivery System, Vol. 21, No. 3 (May 2006), p. 365 is expressly incorporated by reference herein.

### BACKGROUND OF THE INVENTION

1. Field of the Invention

The present invention relates to a microparticle preparation that encapsulates a pharmaceutical agent and a medical composition that contains the microparticle preparation.

2. Description of Related Art

Cisplatin (cis-dichlorodiammineplatinum (II) or CDDP) has been widely used in treatments of a malignant tumor such as a testicular tumor, bladder cancer, ovary cancer, uterine cervix cancer and ureteropelvic junction tumor. The cisplatin is an effective anticancer agent, but can cause severe side-effects such as a neural toxicity, kidney toxicity etc. Accordingly, cisplatin has been administered in a restrictive manner. In order to reduce the side effects of cisplatin while maintaining an anticancer effect, it is important to selectively deliver cisplatin only to a tumor site and to maintain a high concentration of cisplatin. In this manner, cisplatin is administered intracavemously, intraperitoneally and intratumorally.

However, cisplatin in solution is rapidly absorbed into systemic circulation, so that the high concentration of the pharmacological agent can be hardly maintained at a target site.

Consequently, there has been an attempt to use a drug delivery system (DDS) such as a liposome, polymer micelle or a microparticle so as to maintain the high concentration of cisplatin at the target site. A number of researches have been carried out on encapsulating cisplatin by use of a biodegradable polymer. As such examples, refer to Document 1: JP-A-2006-312015 and Document 2: JP-A-2006-248978.

### SUMMARY OF THE INVENTION

However, in the technology of the polymer-coated composition disclosed in Document 1 and the liposome preparation in Document 2, since a large amount of cisplatin is released in an early-phase, cisplatin is not continuously released at a constant concentration.

An object of the present invention is to provide a microparticle preparation that can reduce the early-phase release amount, maintain a release of a pharmaceutical agent over a long period of time and enhance therapeutic efficacy for patients, and a medical composition containing the microparticle preparation.

A microparticle preparation according to an aspect of the present invention contains double-helical DNA, in which a pharmaceutical agent is intercalated into the double-helical DNA.

The double-helical DNA is generally formed by a pair of polynucleotide chains having a right-handed helix. In sugar-phosphate backbones of the pair of polynucleotide chains, a pair of planar bases having structural complementarity protrude toward a center portion of the helix perpendicularly to an axis of the helix, such that the bases are hydrogen-bonded with each other. In a case of a B structure, there is a gap (width and height: approximately 1.1 nm and 0.34 nm respectively) between base pairs of the chains of the DNA. A small molecule having a planar structure can be inserted into the gap, which is called “intercalation (intercalate)”. Such a phenomenon may be promoted by an electric charge and hydrophobic property of the small molecule.

As a material for the double-helical DNA according to the present invention, for example, a fish milt may be advantageously used. Since a fish milt nucleus contains a lot of DNA and has been a discarded material, the milt is an advantageous material for mass-producing the double-helical DNA at low cost. The fish is salmon, herring, trout, cod and the like. After skin, strings and vessels are removed from the milt, the milt is deprived of oil content and refined to obtain the double-helical DNA according to the present invention.

According to the aspect of the present invention, since the microparticle preparation contains a pharmaceutical agent that is intercalated into the double-helical DNA, the microparticle preparation exhibits an excellent slow-release property. Accordingly, for example, a pharmaceutical agent having a severe toxicity can be slowly delivered to a target site.

Particularly, a use of the double-helical DNA derivatized by a ligand having target-cell specificity is advantageous because a specific target site can be selectively cured.

In the microparticle preparation according to the aspect of the present invention, it is advantageous that the pharmaceutical agent is an anticancer agent, and specifically advantageous that the anticancer agent consists of a platinum compound selected from a group consisting of cisplatin, carboplatin, ormaplatin, oxialiplatin and lobaplatin. Particularly, when the microparticle preparation is applied to cisplatin (hereinafter, “CDDP”) that is an anticancer agent having an extremely severe toxicity, the micro preparation can minimize the side effects and continuously provide an anticancer effect to on cancer cells for a long period of time.

As such double-helical DNA, salmon DNA may be advantageously used, which is a biodegradable and nontoxic natural high molecule.

According to the aspect of the present invention, the microparticle preparation can be used as an easily injectable preparation. When the microparticle preparation is provided to medical industries, the microparticle preparation can reduce the side effects entailed by anticancer agent administration, alleviate pains that terminal cancer patients suffer from, and contribute to reduction of medical care costs.

According to the aspect of the present invention, the microparticle preparation may be supported by a pharmaceutically-permissible carrier, so that the microparticle preparation can be used as a medical composition for treating a tumor.

### BRIEF DESCRIPTION OF THE DRAWINGS

FIG. 1 is a scanning electron micrograph showing a morphologic structure of a microparticle according to an aspect of the present invention;

FIG. 2 is a graph showing CDDP release amount according to a first example of the present invention;

FIG. 3 is a graph showing a cytostatic effect of CDDP-DNA MS according to a second example of the present invention;

FIG. 4 is a graph showing weight changes of a mouse according to a third example of the present invention;

FIG. 5 is a graph showing leukocyte number of the mouse according to the third example of the present invention;

FIG. 6 is a graph showing hemoglobin level of the mouse according to the third example of the present invention;

FIG. 7 is a graph showing platelet number of the mouse according to the third example of the present invention;

FIG. 8 is a graph showing blood urea nitrogen amount of the mouse according to the third example of the present invention;

FIG. 9 is a graph showing survival rate of the mouse according to a fourth example of the present invention;

### DETAILED DESCRIPTION OF PREFERRED EMBODIMENT(S)

The present invention will be described below with reference to examples, but it should be noted that the present invention is not limited to the contents of the examples.

### Manufacturing Example

### (1) Method for Preparing Double-Helical DNA by Extracting and Refining

After the fish (salmon) milt underwent a water-rinsing and caustic-soda treatment, the milt underwent a protease treatment and a solid-liquid separation with ethanol to obtain double-helical DNA.

### (2) Manufacturing Method of Microparticle Containing CDDP

A microparticle containing CDDP was manufactured by Solvent Evaporation Method using an O/O emulsion (oil in oil emulsion). Specifically, the microparticte was manufactured by the following method:

500 mg of the double-helical DNA (molecular weight: one million and more) was dissolved in 50 ml of distillated water, which was added with 5 ml of 1 percent-by-mass CDDP solution whose solvent was N,N-dimethylformamide (manufactured by Wako Pure Chemical Industries, Ltd.), and was stirred. Next, the CDDP-DNA mixed solution was added into liquid paraffin containing solbitan monooleat (span 80) of a temperature of 28 degrees Celsius. Being stirred, the CDDP-DNA mixed solution and the liquid paraffin were heated up to 40 degrees Celsius in increments of 2 degrees per ten minutes, and the CDDP-DNA mixed solution and the liquid paraffin were reacted with each other for forty eight hours. The paraffin solution underwent a centrifugal separation at 10000 rpm for ten minutes, and the microparticle was obtained. After being rinsed in n-hexane five times and then in 2-propyl alcohol one time, the microparticle was freeze-dried.

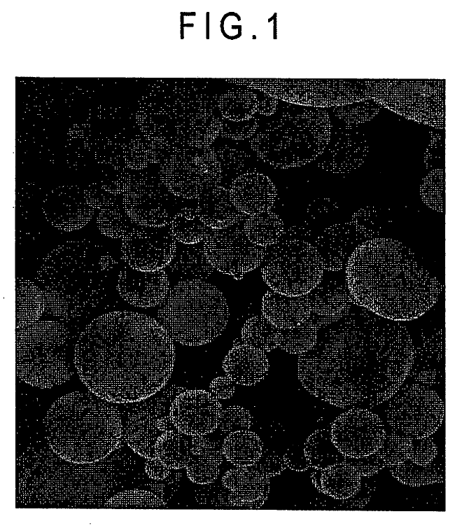

The double-helical DNA prepared in the above-described method (1) was a white and water-soluble material. Containing the CDDP, the microparticle obtained by the Solvent Evaporation Method became a yellow and insoluble material (hereinafter, the microparticle may be referred to as “CDDP-DNA MS”). Such a change suggests that the CDDP was intercalated into a double-helical structure of the DNA such that the CDDP was chemically bonded with the DNA to form a cross-linking. Accordingly, it is considered that the CDDP was stably incorporated into the microparticle. FIG. 1 shows a morphologic structure of the microparticle observed through a scanning electron microscope. The CDDP-DNA MS was a sphere having smooth surfaces without irregularities, of which average diameter was 15 to 25 μm. Further, a product obtained by the method for preparing the microparticle was a dry powder.

### (3) Measurement of CDDP content in CDDP-DNA MS

In order to measure CDDP content in the obtained microparticle, 10 mg of the microparticle was added to 10 ml of nuclease-tris-HCl buffer solution (3 unit/ml, sigma) containing a DNA-degrading enzyme and stirred until the microparticle was completely dissolved. In addition, 10 mg of the CDDP was dissolved in 10 ml of tris-HCl buffer solution, and a solution having a concentration of 0.5 to 2 μg/ml was used as a standard solution. The CDDP content was measured three times per a sample with an atomic absorption spectrometer (Hitachi Model Z-8000).

An atomic absorption spectrum analysis was performed under the following conditions:

Pt lamp: 1=265.9 nm

Slit width: 1.9 A

Carrier gas: argon (1 l/min)

measurement mode: BGC

amount of infusion: 10 μl.

As a result, the CDDP content in the CDDP-DNA MS was 4 percent-by-mass (theoretical figure: 9 percent-by-mass) and an uptake efficiency was 45 percent.

### Example 1

### In Vitro Release Experiment

The CDDP-DNA MS (10 mg) was added to 10 ml of tris-HCl buffer solution (pH 7.4) containing 0.01 percent-by-mass Tween 80. Being oscillated, the buffer solution was incubated at a temperature of 37 degrees Celsius respectively for two hours, one day, three days, five days and seven days. 5 ml of the buffer solution was extracted at each point, and a Pt amount in the solution was measured by measuring absorbency under 265.95 nm with an atomic absorption spectrometer. The measurement was performed independently three times.

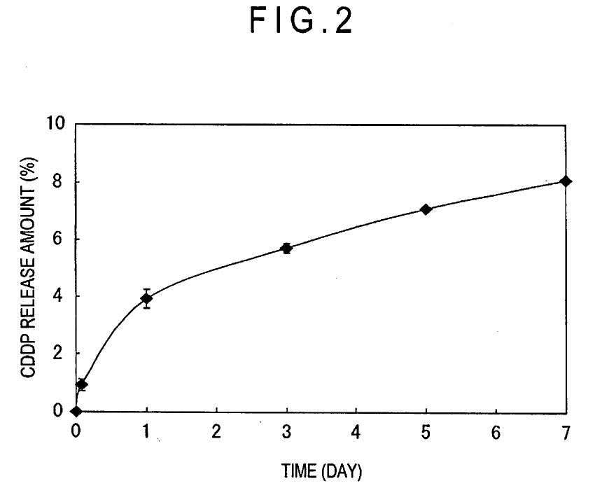

FIG. 2 shows the CDDP amount released from the CDDP-DNA MS. A CDDP release experiment was conducted under an environment with no DNA-degrading enzyme, and the CDDP content released from the CDDP-DNA MS was measured for 7 days. As a result, a released amount of the CDDP was confirmed to be 8% of the amount incorporated into the microparticle. Because the early-phase release amount was observed not large, a slow-release property of the CDDP was confirmed. It is considered that the above result was observed because the DNA degradation was delayed under the environment with no enzyme, such that the release of the CDDP from a DNA-CDDP complex was suppressed. A large amount of early-phase release from the microparticle means that an agent surrounding the microparticle surface is released due to diffusion. Since the early-phase release amount from the microparticle according to the present invention is not large, it is understood that the CDDP is stably bonded with the DNA.

### Example 2

### In vitro Cellular Toxicity Experiment

Using mouse-derived colon cancer cells (colon-26), a toxicity of the microparticle to cancer cells was experimented. Specifically, the experiment was conducted according to the following method:

The colon-26 cells were disseminated on a 96 well-plate at 1′104 concentration and cultured for twenty four hours. Then, the cells were treated with free CDDP (free CDDP: 1 μg/ml, 5 μg/ml, 10 μg/ml), DNA only (5 mg/ml) and the CDDP-DNA MS (CDDP-MS: 1 mg/ml, 10 mg/ml) and cultured for forty eight hours. Cellular toxicity was examined by measuring absorbency under 450 nm with a CCK-8 kit (Dojindo, Kumamoto, Japan), the CCK-8 kit measuring formazan substitution of tetrazolium based on an intercellular enzyme.

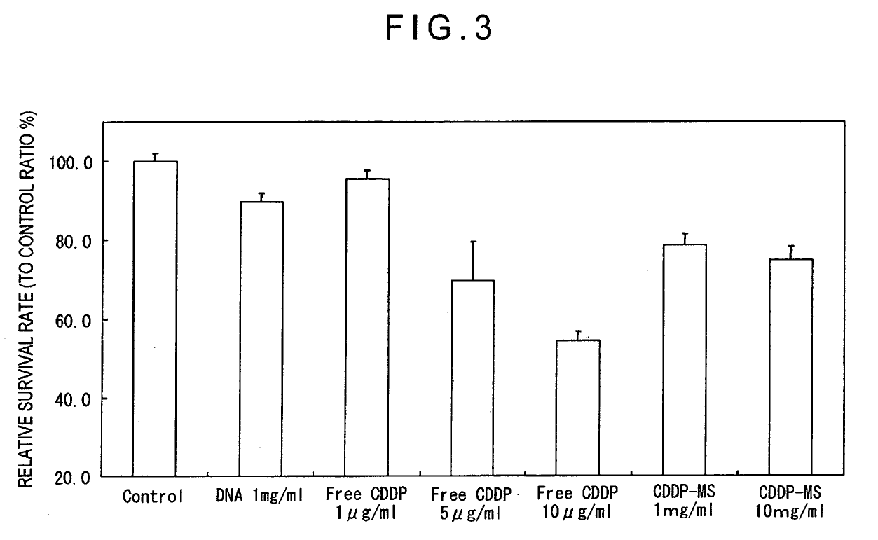

FIG. 3 shows cytostatic effect that the free CDDP and CDDP-DNA MS made on colon cancer cells of colon-26 after the forty eight hour treatment.

Cell growth was slightly depressed in the colon cancer cells of colon-26 treated with the DNA only. However, since no effect was made on the cell-growth in an experiment of toxicity to fibroblast L-929, the cell-growth depression in colon-26 is considered not to have been induced by the toxicity.

The cell group having undergone the 10 μg/ml free CDDP treatment showed the cytostatic effect of approximately 50 percent. In the cells treated with the CDDP-DNA MS at concentrations of 1 mg/ml and 10 mg/ml respectively, the cell growth was significantly depressed as compared with a control (standard sample). However, the concentration difference of the CDDP-DNA MS showed no significant difference in the cell-growth depression effect. It is now clear that the CDDP released from the CDDP-DNA MS retains activity in depressing the cell-growth.

### Example 3

### In Vivo Toxicity Experiment

As an experimental animal, six-week-old BALB/c male mice (weight 25 g, Shimizu Laboratory Supplies Co., Ltd.) was kept under an environment where a constant temperature and humidity was maintained (temperature: 24 degrees Celsius +/−2 degrees, humidity: 55+/−10%). The mice were divided into control group, a CDDP-DNA MS group and a free CDDP group, each including five mice. During an experiment, the mice were allowed to access to feed and water without restriction. The CDDP-DNA MS, free CDDP and DNA only were respectively suspended in normal saline containing 0.01 percent by mass Tween 80, and each solution was adjusted.

Normal saline was set as the control. The free CDDP was prepared to have concentrations respectively of 5 mg/kg, 10 mg/kg and 20 mg/kg, and the mice in the free CDDP group were intraperitoneally administered with the free CDDP one time for each concentration. The CDDP-DNA MS was prepared to have CDDP concentrations respectively of 20 mg/kg and 40 mg/kg, and the mice in the CDDP-DNA MS group were intraperitoneally administered with the CDDP-DNA MS one time for each concentration. Then, mice conditions were observed, whether or not the mice were alive was checked, and weight of the mouse was measured. The mice were sacrificed in 30 days after the administration. Blood was taken from hearts of the mice and a hematological assessment and biochemical examination of blood were conducted. Further, after dissection, internal organs were macroscopically observed.

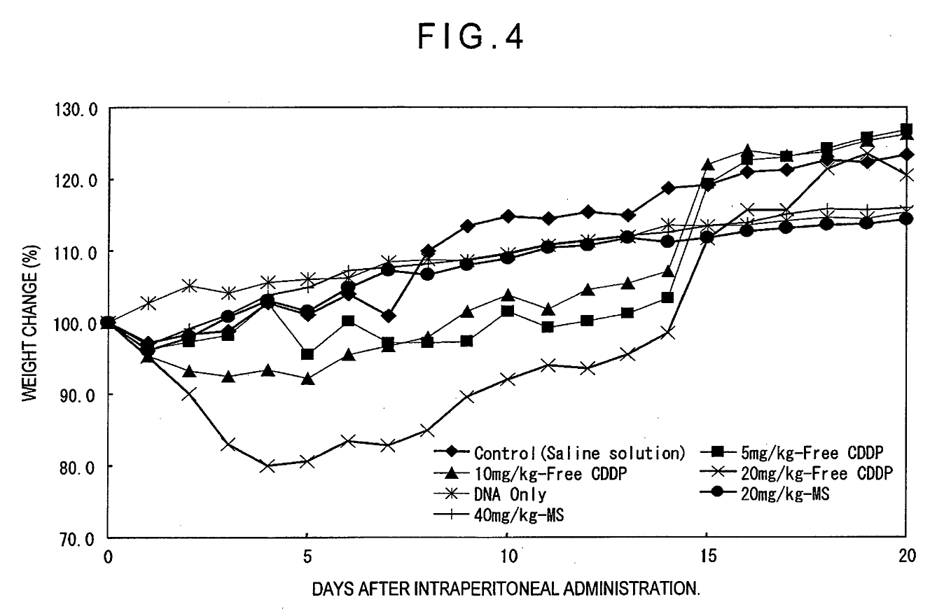

FIG. 4 shows weight changes of the mice during twenty days after the intraperitoneal administration of the free CDDP and CDDP-DNA MS. In the free CDDP group, irrespective of the concentration, weight reduction was observed from the next day of the administration and a maximum weight reduction was observed on a fourth or fifth day after the administration. Especially the mice administered with the free CDDP of the mg/kg concentration showed 20-percent weight reduction. The weight was gradually increased from the fifth day after the administration when the minimum weight was observed, and approached a value of the control group on a fifteenth day. On the other hand, in the CDDP-DNA MS group, no drastic weight change was confirmed, and the weight change pattern was similar to that of the control group. Based on the above observation, toxicity entailed by the one-time administration of the free CDDP was confirmed in the form of the weight reduction during an early phase of the administration. However, it is considered that no toxicity was entailed by the CDDP-DNA MS administration.

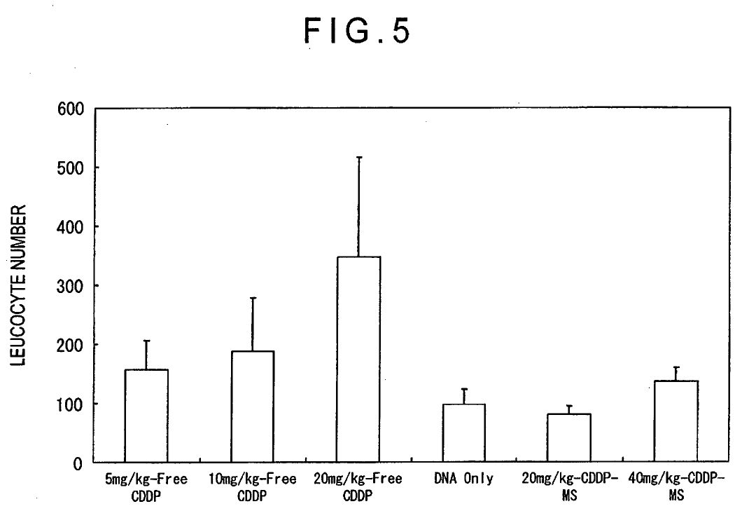

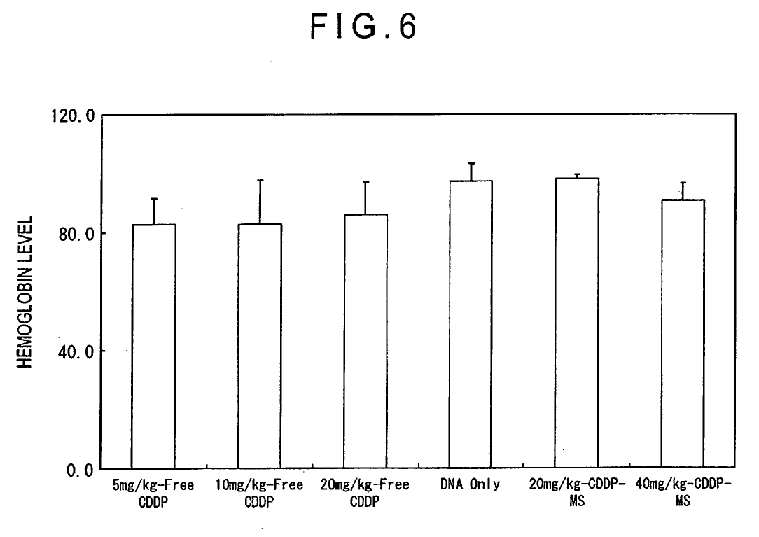

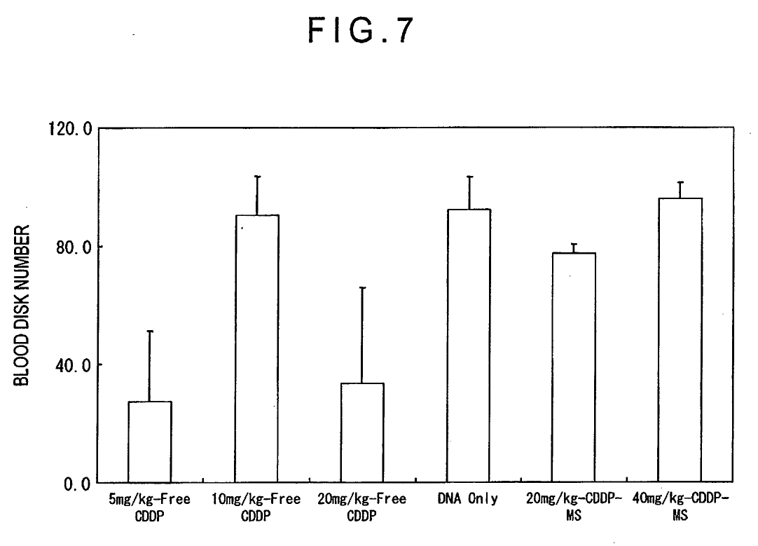

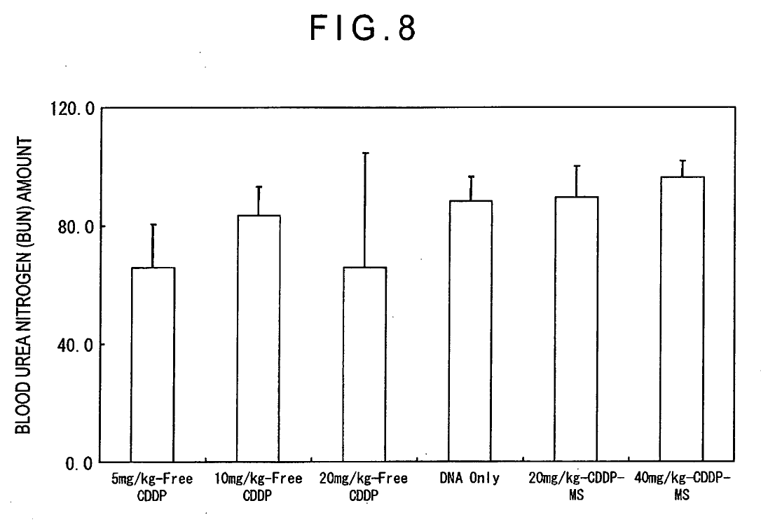

The mice were sacrificed on a twentieth day after the agent administration, and FIGS. 5 to 8 show results of the hematologic assessment and biochemical examination of blood. With respect to leukocyte number (FIG. 5), the free CDDP group showed a tendency to decrease, but the CDDP-DNA MS group showed no difference from that in the control group. With respect to hemoglobin level (FIG. 6), there was no significant difference between the CDDP-DNA MS group and the free CDDP group. With respect to blood disk number (FIG. 7), the free CDDP group showed a significant decrease as compared with the control group, but the CDDP-DNA MS group showed no significant change. According to the results of the hematologic assessment, toxicity entailed by the free CDDP can be confirmed even after thirty days from the administration. A blood urea nitrogen measurement was conducted so as to check nephrotoxicity, which is a representative side effect of the CDDP. FIG. 8 shows results of the blood urea nitrogen measurement. Although the CDDP-DNA MS group showed a slight increase, there was no significant difference among each of the groups.

Based on the above results, it is understood that the CDDP-DNA MS administration entails much less toxicity than the free CDDP administration.

### Example 4

### In Vivo Experiment of Anticancer Treatment

As an experimental animal, a six-week-old BALB/c male mouse (weight 25 g, Shimizu Laboratory Supplies Co., Ltd.) was selected. Based on weight measured on a date of cancer implanting, the mice were divided by stratified randomization method into a control group, a CDDP-DNA MS group and a free CDDP group such that each group included seven mice. In the experiment, the same agent treatment was performed as in the toxicity experiment. Specifically, the experiment was conducted in the following methods:

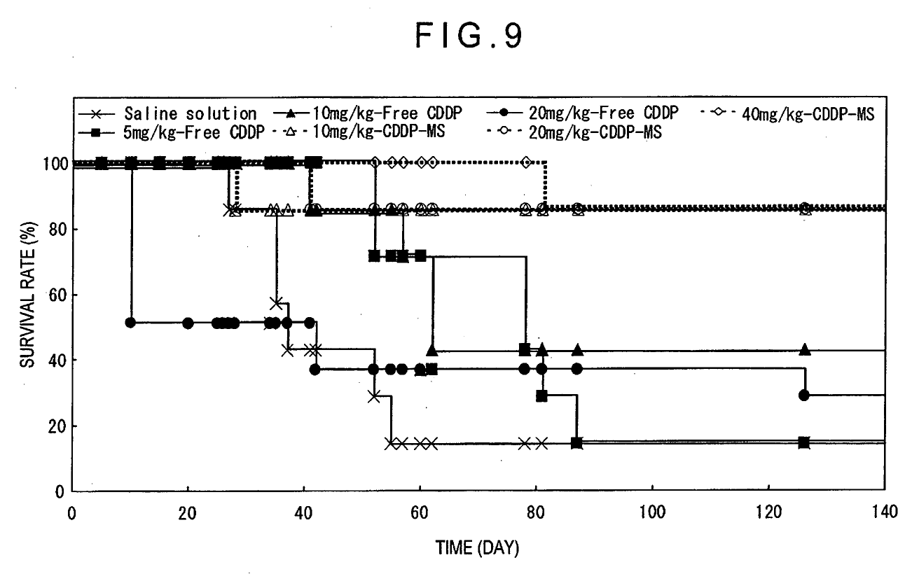

Mouse-derived colon cancer (colon-26 adenocarcinoma) cells (1′106) were intraperitoneally implanted in the mice to provide cancer-bearing mice. In six days after the cancer implanting, agents (the free CDDP and CDDP-DNA MS) were intraperitoneally administered. Weight changes and survival rate were observed from the day of the administration. In FIG. 9, results are represented by a mouse-survival rate curve.

After the agent administration, the free CDDP group showed weight reduction and appearance changes due to CDDP toxicity. In the free CDDP group in which 20 mg/kg of CDDP was administered, a minimum weight was observed on a fifth day after the administration and a half number of the group eventually died. The death is considered to be caused by the CDDP toxicity.

On the other hand, neither weight reduction nor appearance change was observed in the CDDP-DNA MS group. In the free CDDP group, luster of the mouse hair was lost after the CDDP administration.

The appearance changes due to cancer formation were observed from a thirtieth day after the administration. In the free CDDP group, the mice started to die of cancer after a fortieth day. The concentration difference between the free CDDP 5 mg/kg and 10 mg/kg showed no significant difference in the anticancer effect On the other hand, in the CDDP-DNA MS group, it was obvious from an appearance observation that a tumor formation was depressed, and the CDDP-DNA MS showed a significant difference in life prolongation effect as compared with the control and free CDDP groups.

From the results, it is understood that the administration of the CDDP-DNA MS provides an excellent anticancer effect.

What is claimed is:

1. A microparticle preparation containing double-helical DNA, wherein
a pharmaceutical agent is intercalated into the double-helical DNA.

2. The microparticle preparation according to claim 1, wherein
the pharmaceutical agent is an anticancer agent.

3. The microparticle preparation according to claim 2, wherein
the anticancer agent consists of at least one platinum compound selected from a group consisting of cisplatin, carboplatin, ormaplatin, oxialiplatin and lobaplatin.

4. A medical composition for treating a tumor, comprising;
the microparticle preparation according to claim 1; and
a pharmaceutically-permissible carrier.

5. A medical composition for treating a tumor, comprising;
the microparticle preparation according to claim 2; and
a pharmaceutically-permissible carrier.

6. A medical composition for treating a tumor, comprising;
the microparticle preparation according to claim 3; and
a pharmaceutically-permissible carrier.
